(19) United States
(12) Patent Application Publication
(10) Pub. No.: US 2018/0310867 A1
(43) Pub. Date: Nov. 1, 2018

(54) SYSTEM AND METHOD FOR STRESS LEVEL MANAGEMENT

(71) Applicant: WELLBE DIGITAL LTD, Nazereth (IL)

(72) Inventors: Tsachi SIVAN, Ramat Hasharon (IL); Doron LIBESHTEIN, Kfar Neter (IL); Yariv Avraham AMOS, Tzorit (IL)

(73) Assignee: WELLBE DIGITAL LTD., Nazereth (IL)

(21) Appl. No.: 15/772,533

(22) PCT Filed: May 15, 2016
(86) PCT No.: PCT/IL16/50507

§ 371 Date: May 1, 2018

Related U.S. Application Data
(60) Provisional application No. 62/281,562, filed on Jan. 21, 2016.

(51) Int. Cl. A61B 5/16; A61B 5/024; A61B 5/00

(52) U.S. Cl. CPC A61B 5/165; A61B 5/681; A61B 5/024

(57) ABSTRACT

A system comprising: at least one sensor, comprised within a wireless wearable electronic device, the sensor configured to sense, over a given time period, information enabling determination of a Heart Rate Variability (HRV) of a wearer of the wireless wearable electronic device; and one or more processing units configured to: obtain, from the sensor, the information enabling determination of the HRV of the wearer; determine, using the information, the HRV of the wearer; calculate, utilizing the HRV, a stress score indicative of a stress level of the wearer.

### TECHNICAL FIELD

The invention relates to a system and method for stress level management.

### BACKGROUND

Various stress level measurement devices exist nowadays, however such devices are usually operated by trained medical personnel, in a pre-schedule session during which the patient is aware of the fact that his stress level is measured. As a result: (1) the patient's awareness of the measure can influence the measured result; (2) the patient can learn his stress level at the time of the measure but not at other times where the patient may be more stressed (3) the patient does not have any means of knowing when his stress level is too high (and thus, also in cases where the stress level compromises his health, he cannot take any action to reduce his stress level. In addition, the current stress level measurement devices do not associate the measured stress levels with corresponding environmental data (information of locations of the patient, meetings the patient attended, people the patient met, etc.), which can explain fluctuations in the stress level throughout a monitored time period.

There is thus a need in the art for a new method and system for stress level management.

### CROSS-REFERENCE TO RELATED APPLICATIONS

This application claims priority to U.S. Provisional Application No. 62/281,562, filed Jan. 21, 2016 which is incorporated herein by reference.

### GENERAL DESCRIPTION

In accordance with a first aspect of the presently disclosed subject matter there is provided a system comprising: at least one sensor, comprised within a wireless wearable electronic device, the sensor configured to sense, over a given time period, information enabling determination of a Heart Rate Variability (HRV) of a wearer of the wireless wearable electronic device; and one or more processing units configured to: obtain, from the sensor, the information enabling determination of the HRV of the wearer; determine, using the information, the HRV of the wearer; calculate, utilizing the HRV, a stress score indicative of a stress level of the wearer.

In some cases, the HRF includes at least one of: a High Frequency HRV (HRV-HF); a Low Frequency HRV (HRV-LF); a combined measure of the HRV-HF and the HRV-LF.

In some cases, at least one of the processing units is further configured to store the stress score and a time associated with the stress score in a data repository comprising a plurality of previously calculated stress scores, each indicative of a corresponding stress level of the wearer at a corresponding time.

In some cases, the system comprises at least one display and at least one of the processing units is further configured to retrieve at least one previously calculated stress score of the previously calculated stress scores from the data repository, and present the at least one previously calculated stress score on the display, optionally along with at least one corresponding environmental data associated therewith.

In some cases, the corresponding environmental data includes one or more of: data indicative an event the wearer attended at the corresponding time of the corresponding previously calculated stress score; data indicative of participants of the event; and data indicative of a location of the wearer at the corresponding time of the corresponding previously calculated stress score.

In some cases, at least one of the processing units is further configured to alert the wearer when the stress score exceeds a threshold.

In some cases, the threshold is pre-defined or dynamically determined by at least one of the processing units utilizing previously calculated stress scores each indicative of a corresponding stress level of the wearer.

In some cases, the wireless wearable device further comprises a motion detection system configured to obtain an indication of a motion status of the wireless wearable device and wherein the information enabling determination of the HRV of the wearer is obtained when the indication indicates that the wireless wearable device is not moving.

In some cases, the motion detection system comprises at least one accelerometer.

In some cases, the determine comprises: dividing the given time period to a plurality of time windows; calculating, for each time window, a corresponding time window HRV; and selecting the maximal time window HRV, indicative of the lowest stress level of the wearer during the given time window.

In some cases, each pair of subsequent time windows of the plurality of time windows at least partially overlap.

In some cases, the wireless wearable electronic device is worn on a wrist of the wearer.

In some cases, at least a first processing unit of the processing units is comprised within the wireless wearable electronic device, and at least a second processing units of the processing units is external to the wireless wearable electronic device.

In some cases, the stress score is calculated also utilizing at least one of the following parameters: an average interval between heartbeats of the user during the given time period; a standard deviation of intervals between heartbeats of the user during the given time period.

In accordance with a second aspect of the presently disclosed subject matter there is provided a method comprising: obtaining from at least one sensor, comprised within a wireless wearable electronic device, information sensed during a given time period, the information enabling determination of a Heart Rate Variability (HRV) of a wearer of the wireless wearable electronic device; determining, using the information, the HRV of the wearer; calculating, utilizing the HRV, a stress score indicative of a stress level of the wearer.

In some cases, the HRF includes at least one of: a High Frequency HRV (HRV-HF); a Low Frequency HRV (HRV-LF); a combined measure of the HRV-HF and the HRV-LF.

In some cases, the method further comprises storing the stress score and a time associated with the stress score in a data repository comprising a plurality of previously calculated stress scores, each indicative of a corresponding stress level of the wearer at a corresponding time.

In some cases, the method further comprises retrieving at least one previously calculated stress score of the previously calculated stress scores from the data repository, and presenting the at least one previously calculated stress score on a display, optionally along with at least one corresponding environmental data associated therewith.

In some cases, the corresponding environmental data includes one or more of: data indicative an event the wearer attended at the corresponding time of the corresponding previously calculated stress score; data indicative of participants of the event; and data indicative of a location of the wearer at the corresponding time of the corresponding previously calculated stress score.

In some cases, the method further comprises alerting the wearer when the stress score exceeds a threshold.

In some cases, the threshold is pre-defined or dynamically determined utilizing previously calculated stress scores each indicative of a corresponding stress level of the wearer.

In some cases, the method further comprises obtaining, from a motion detection system comprised within the wireless wearable device, an indication of a motion status of the wireless wearable device and wherein the information enabling determination of the HRV of the wearer is obtained when the indication indicates that the wireless wearable device is not moving.

In some cases, the motion detection system comprises at least one accelerometer.

In some cases, the determining comprises: dividing the given time period to a plurality of time windows; calculating, for each time window, a corresponding time window HRV; and selecting the maximal time window HRV, indicative of the lowest stress level of the wearer during the given time window.

In some cases, each pair of subsequent time windows of the plurality of time windows at least partially overlap.

In some cases, the wireless wearable electronic device is worn on a wrist of the wearer.

In some cases, the stress score is calculated also utilizing at least one of the following parameters: an average interval between heartbeats of the user during the given time period; a standard deviation of intervals between heartbeats of the user during the given time period.

In accordance with a third aspect of the presently disclosed subject matter there is provided a non-transitory computer readable storage medium having computer readable program code embodied therewith, the computer readable program code, executable by at least one processor of a computer to perform a method comprising: obtaining from at least one sensor, comprised within a wireless wearable electronic device, information sensed during a given time period, the information enabling determination of a Heart Rate Variability (HRV) of a wearer of the wireless wearable electronic device; determining, using the information, the HRV of the wearer; calculating, utilizing the HRV, a stress score indicative of a stress level of the wearer.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a schematic illustration of an environment of a system for stress level management, in accordance with the presently disclosed subject matter;

FIG. 2 is a block diagram schematically illustrating one example of a system for stress level management, in accordance with the presently disclosed subject matter;

FIG. 3 is a flowchart illustrating one example of a sequence of operations carried out for stress level management, in accordance with the presently disclosed subject matter; and

FIG. 4 is a flowchart illustrating one example of a sequence of operations carried out for calculating a Heart Rate Variability (HRV) of a user of the stress level management system, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “obtaining”, “determining”, “calculating”, “storing”, “retrieving”, “presenting”, “alerting”, “dividing”, “selecting” or the like, include action and/or processes of a computer that manipulate and/or transform data into other data, said data represented as physical quantities, e.g. such as electronic quantities, and/or said data representing the physical objects. The terms “computer”, “processor”, and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

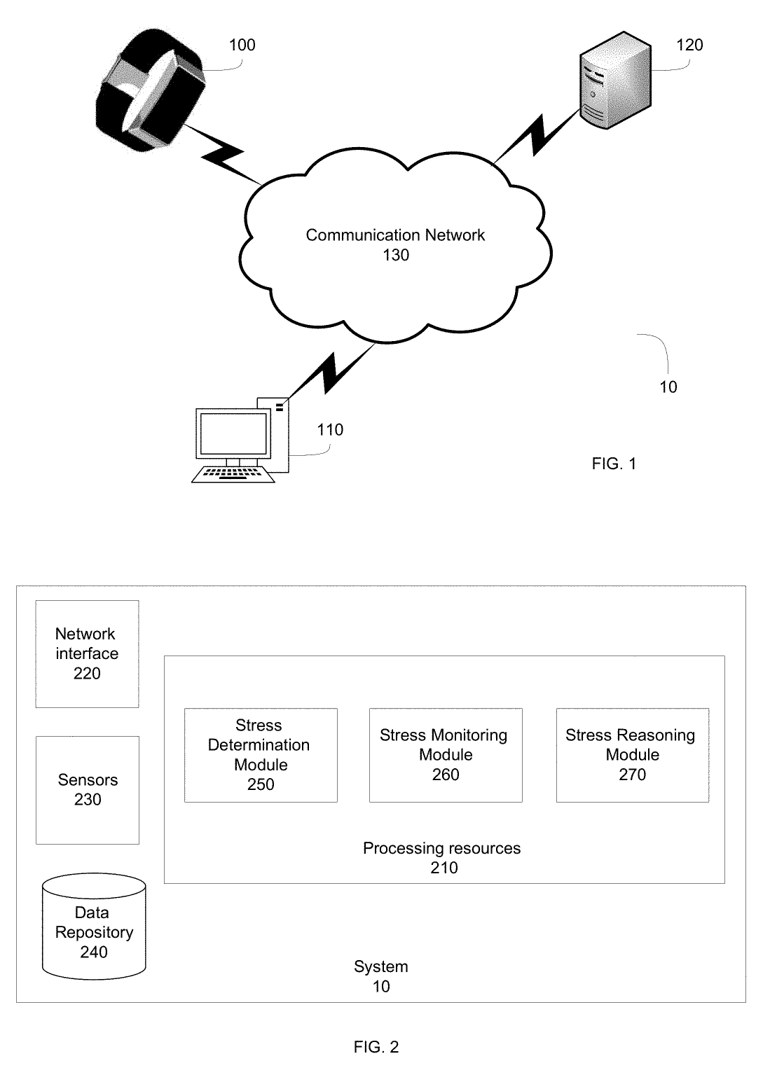

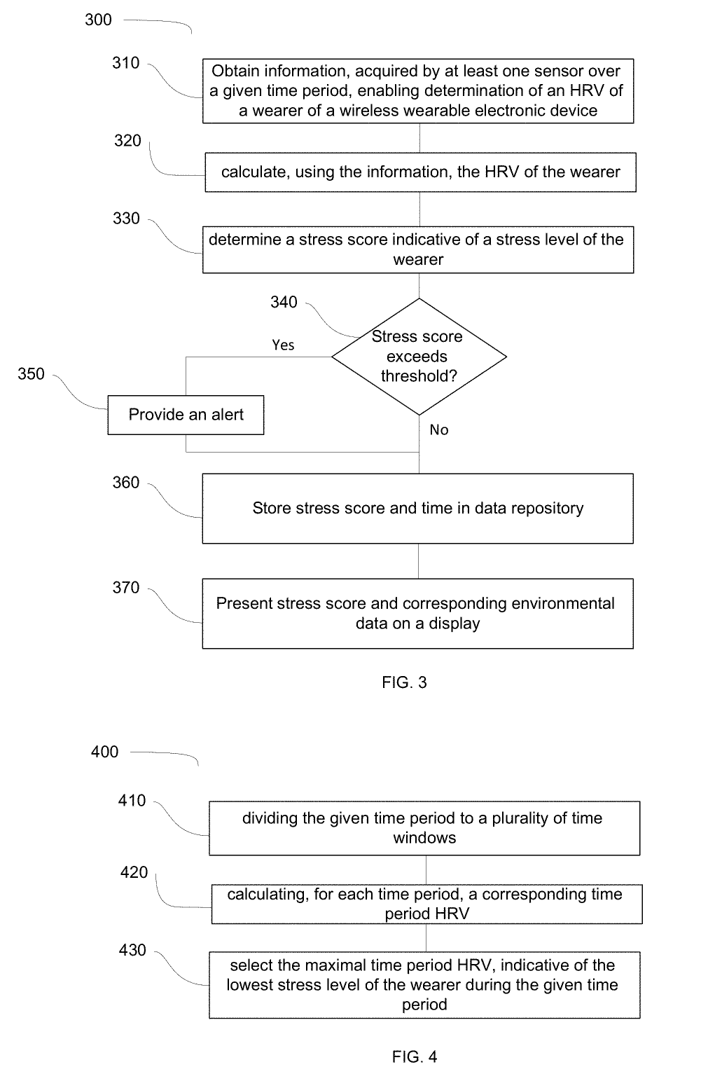

In embodiments of the presently disclosed subject matter, fewer, more and/or different stages than those shown in FIGS. 3 and 4 may be executed. In embodiments of the presently disclosed subject matter one or more stages illustrated in FIGS. 3 and 4 may be executed in a different order and/or one or more groups of stages may be executed simultaneously. FIGS. 1 and 2 illustrate a general schematic of the system architecture in accordance with an embodiment of the presently disclosed subject matter. Each module in FIG. 2 can be made up of any combination of software, hardware and/or firmware that performs the functions as defined and explained herein. The modules in FIG. 2 may be centralized in one location or dispersed over more than one location. In other embodiments of the presently disclosed subject matter, the system may comprise fewer, more, and/or different modules than those shown in FIG. 2.

Bearing this in mind, attention is drawn to FIG. 1, showing a schematic illustration of a system for stress level management, in accordance with the presently disclosed subject matter.

According to certain examples of the presently disclosed subject matter, the system 10 includes a wireless wearable electronic device 100, configured to be worn by a user of the system whose stress level determination is required. In some cases, the wireless wearable electronic device 100 can be design to be worn on the user's wrist. In other cases, the wireless wearable electronic device 100 can be worn in another manner on other locations of the user's body (e.g. the user's chest, the users finger, etc.), as long as it is capable of sensing information enabling determination of a stress level of the user wearing it.

The information that enables determination of the stress level of the user can be acquired by one or more sensors comprised within the wireless wearable electronic device 100, as further detailed herein, inter alia with reference to FIG. 2. One Exemplary measure based on which a stress level of the user can be determined is the user's Heart Rate Variability (HRV) that can be itself determined by detecting the intervals between heartbeats. The HRV can be calculated using the measured high frequency heartbeat activity (also referred to herein as HRV-HF) (e.g. within the range of 0.15-0.5 Hz), or using the measured low frequency heartbeat activity (also referred to herein as HRV-LF) (e.g. within the range of 0.05-0.15 Hz), or using a combination of the low frequency and high frequency heartbeat activity.

It is to be noted that the Heart Rate Variability can be evaluated by a number of methods including time domain and/or frequency domain. With both methods the heart rate at any point in time and/or the interval between successive normal heartbeat complexes is defined. It is to be further noted that the HRV is determined using the Normal to Normal (NN) intervals (the interval between two consecutive normal heartbeats). It is to be still further noted that the determination whether a heartbeat is a normal heartbeat can be performed in accordance with the teachings of Logier R et al., IEEE EMBS, 26 Sep. 2004, pp 3937-3940.

Once the NN interval is defined one or more of the following time domain HRV parameters can be extracted:
a. Mean [milliseconds]—The average of all NN intervals measured during the examined time period;
b. SDNN [milliseconds]—the standard deviation of all NN intervals measured during the examined time period (this parameter can be replaced by the variance of the normal to normal beats (VarNN)).

In some cases, the frequency domain parameters are based on a spectral estimation of all NN intervals after interpolation, e.g. using Berger method (see Berger, R., Akselrod, S., Gordon, D., and Cohen, R. “An efficient algorithm for spectral analysis of HRV”, IEEE-Trans-Biomed-Eng. 1986; 33 (9): 900-4). Additionally or alternatively, one or more of the following frequency domain parameters can be extracted utilizing the NN intervals:
a. HRV-HF [milliseconds²]—power in high frequency range (e.g. 0.15-0.5 Hz).
b. HRV-LF [milliseconds²]—power in high frequency range (e.g. 0.05-0.15 Hz).

It is to be noted that other parameter, such normalized HRV-HF, normalized HRV-LF and total energy, can be calculated as well.

In some cases, the entire system 10 can be comprised within the wireless wearable electronic device 100. However, in other cases, the system 10 can further include at least one other computing device 110 (e.g. a desktop computer, a laptop computer, a tablet computer, a smartphone, etc.) capable of wirelessly communicating with the wireless wearable electronic device 100 directly (e.g. utilizing Bluetooth/ZigBee or similar connection enabling components) or over a communication network 130 such as an Intranet/Internet (e.g. utilizing a WiFi client, etc.). In such cases, the system 10 can be configured so that the other computing device 110 receives data (either raw data from the sensors, or data that has been processed to some extent) from the wireless wearable electronic devices 100 and performs at least part of the processing of the system 10 as further detailed herein.

In some cases, the system 10 can further comprise one or more centralized servers 120 configured to store data relating to a plurality of users, including previously calculated stress scores, each indicative of a corresponding stress level of a corresponding user wearing a corresponding wireless wearable electronic device 100. In such cases, the centralized servers 120 can obtain the stress scores and/or other data, via the communication network 130. Such data can be sent to the centralized servers 120 by one or more wireless wearable electronic devices 100 directly, or through the other computing devices 110 that are in communication with the wireless wearable electronic devices 100.

It is to be noted that in some cases one or more of the other computing device 110 can be one or more of the centralized servers 120 and vice versa.

Attention is now drawn to FIG. 2, a block diagram schematically illustrating one example of a system for stress level management, in accordance with the presently disclosed subject matter.

As indicated herein, in some cases the system 10 can be fully comprised within the wireless wearable electronic device 100. In such cases, the wireless wearable electronic device 100 can comprise one or more sensors 230, a data repository 240, and one or more processing resources 210.

The one or more sensors 230 can be sensors capable of acquiring information that enables determination of the stress level of the user. As indicated herein, one Exemplary measure based on which a stress level of the user can be determined is the user's Heart Rate Variability (HRV) that can be itself determined by detecting the intervals between normal heartbeats and optionally performing various mathematical operations thereon (e.g. as detailed herein with respect to FIG. 1). In such cases, the sensors 230 can include electrocardiogram which record the electrical activity of a subject and processed it in order to detect the RR interval, piezoelectric sensor for heartbeat measurements, and/or light based sensors capable of detecting changes in blood vessels (such as a pulse oximeter capable of obtaining a photoplethysmogram) that are indicative of heartbeats, a radar capable of detecting changes in the volume of blood within an artery of the user, etc.

The data repository 240 (e.g. a database, a memory including Read Only Memory—ROM, Random Access Memory—RAM, or any other type of memory, etc.) is configured to store data, including, inter alia, raw readings from the sensors, stress scores calculated by the system 10, times associated with the stress score (e.g. an indication of the date and time of day at which the stress score was determined), general and/or user-specific stress scores thresholds, environmental data (e.g. data indicative of event the user attended at various dates and times of day, data indicative of participants of such events, data indicative of locations of the user at various times, etc.), etc. In some cases, data repository 240 can be further configured to enable retrieval and/or update and/or deletion of the data stored thereon.

The one or more processing resources 210 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant system 10 resources and for enabling operations related to system 10 resources.

The one or more processing resources 210 can comprise one or more of the following modules: stress determination module 250, stress monitoring module 260 and stress reasoning module 270.

According to some examples of the presently disclosed subject matter, stress determination module 250 can be configured to determine a stress level of the user wearing the wireless wearable electronic device 100, as further detailed herein, inter alia with reference to FIGS. 3 and 4.

According to some examples of the presently disclosed subject matter, stress monitoring module 260 can be configured to monitor that the stress level of the user wearing the wireless wearable electronic device 100 does not exceed a threshold, and if so—issue an alert to the user, as further detailed herein, inter alia with reference to FIG. 3.

As indicated herein, in some cases, the system 10 further includes at least one other computing device 110 (e.g. a desktop computer, a laptop computer, a tablet computer, a smartphone, etc.) capable of wirelessly communicating with the wireless wearable electronic device 100, and capable of performing at least part of the processing of the system 10. In such cases, the wireless wearable electronic device 100 and the at least one other computing device 110 further comprise a network interface 220 (e.g. a Bluetooth connection enabling component, a ZigBee connection enabling component, a WiFi client capable of wirelessly connecting to the communication network 130, etc.), enabling connecting the wireless wearable electronic device 100 to the other computing device 110. In some cases, the connection is a direct connection (e.g. using Bluetooth or ZigBee, etc.), whereas in other cases the connection is over a communication network 130 (e.g. using WiFi clients). The connection enables the wireless wearable electronic device 100 and the other computing device 110 to exchange data, as detailed herein, inter alia with reference to FIG. 3.

As indicated herein, in some cases, the system 10 further includes one or more centralized servers 120 (e.g. a desktop computer, a laptop computer, a tablet computer, a smartphone, etc.) capable of wirelessly communicating with the wireless wearable electronic device 100 and/or with the other computing device 110, and capable of performing at least part of the processing of the system 10. In such cases, also the centralized servers 120 comprise a network interface 220 (e.g. a Bluetooth connection enabling component, a ZigBee connection enabling component, a WiFi client capable of wirelessly connecting to the communication network 130, etc.), enabling connecting the wireless wearable electronic device 100 and/or the other computing device 110 to the centralized servers 120. In some cases, the connection is a direct connection (e.g. using Bluetooth or ZigBee, etc.), whereas in other cases the connection is over a communication network 130 (e.g. using WiFi clients). The connection enables the wireless wearable electronic device 100 and/or the other computing device 110 to exchange data with the centralized servers 120, as detailed herein, inter alia with reference to FIG. 3.

It is to be noted that in cases where the system 10 is not fully comprised within the wireless wearable electronic device 100, the data repository 240 can be located on the other computing device 110, or on the centralized servers 120. Additionally, or alternatively, the data repository 240 can be distributed between the wireless wearable electronic device 100 and/or the other computing device 110 and/or the centralized servers 120.

It is to be further noted that in cases where the system 10 is not fully comprised within the wireless wearable electronic device 100, the processing resources 210 can be distributed between the wireless wearable electronic device 100 and/or the other computing device 110 and/or the centralized servers 120. In such cases, the processing performed after the information enabling determination of the HRV of the wearer is obtained from the sensors 230 (by a processing resource comprised within the wireless wearable electronic device 100), as further detailed herein inter alia with reference to FIGS. 3 and 4, can be performed by the processing resource of the wireless wearable electronic device 100 and/or the processing resource of the other computing device 110 and/or the processing resource of the centralized servers 120.

Turning to FIG. 3, there is shown a flowchart illustrating one example of a sequence of operations carried out for stress level management, in accordance with the presently disclosed subject matter.

According to some examples of the presently disclosed subject matter, system can be configure to perform a stress monitoring process 300, e.g. utilizing the stress monitoring module 250.

For that purpose, system 10 can be configured to obtain information acquired by the sensors 230 over a given period of time (e.g. one minute, two minutes, three minutes, four minutes, five minutes, etc.), the information enabling determination of the HRV of the user wearing the wireless wearable electronic device 100 (block 310). Utilizing the obtained information, obtained at block 310, the system 10 can determine the HRV of the wearer (block 320), e.g. as further detailed herein with reference to FIGS. 1 and 4.

Utilizing the HRV determined at block 320, system 10 can be configured to calculate a stress score indicative of a stress level of the user wearing the wireless wearable electronic device 100 (block 330). In this respect it is to be noted that low HRV values indicate of a high stress level, and high HRV values indicate of a low stress level. In some cases, the stress score calculation includes performing a random forest regression analysis of the values of the HRV parameters (e.g. mean, SDNN, HRV-HF, HRV-LF, as detailed with respect to FIG. 1) obtained at block 320, with respect to a gold standard stress score, such as Holmes Rahe Stress Scale (HRSS).

In some cases, the system 10 can further utilize the stress monitoring module 260 to check if the stress score calculated at block 330 exceeds a threshold (block 340). The threshold can be a general pre-determined threshold, or it can be a pre-determined user-specific threshold, or it can be a dynamic user-specific threshold (e.g. calculated based on previous stress scores calculated for the specific user). If the stress score exceeds the threshold, the system 10 can be configured to provide the user with an alert (block 350). The alert can be provided by the wireless wearable electronic device 100 (e.g. utilizing a vibrating element that vibrates the wireless wearable electronic device 100 and/or using a speaker of the wireless wearable electronic device 100 that generates a certain sound, etc.). Additionally, or alternatively, the alert can be provided by the other computing device 110 (e.g. utilizing a vibrating element that vibrates the other computing device 110 and/or using a speaker of the other computing device 110 that generates a certain sound, or, in cases where the other computing device 110 is a smartphone, by sending a text message to the user's phone number, etc.). In some cases, the alert can be accompanied by a recommendation on actions to be taken to reduce the stress level. In some cases, the system 10 can re-evaluate the user's stress score to make sure that the user's stress score returned to a level that does not breach the threshold after taking the recommended action.

If the threshold is not exceeded, or after providing the alert to the user, the system 10 can be further configured to store the stress score calculated at block 330, optionally with a timestamp indicative of the date and time the stress score was calculated, in the data repository 240 (block 360).

In some cases, the system 10 can also utilize the stress reasoning module 270 to retrieve the stress score (or another previously calculated stress score) from the data repository for displaying it on a display of the wireless wearable electronic device 100 and/or of the other computing device 100, optionally along with corresponding environmental data (block 370). Such environmental data can include, for example, one or more of:
a. data indicative an event the user wearing the wireless wearable electronic device 100 attended when the information of block 310 was acquired by the sensors 230; It is to be noted that such data can be obtained from one or more calendars associated with the user wearing the wireless wearable electronic device 100, such as Google Calendar, Microsoft Office Calendar, etc.
b. data indicative of participants of the event; It is to be noted that such data can be obtained from one or more calendars associated with the user wearing the wireless wearable electronic device 100, such as Google Calendar, Microsoft Office Calendar, etc.
c. data indicative of a location of the user wearing the wireless wearable electronic device 100 when the information of block 310 was acquired by the sensors 230. It is to be noted that in some cases the location of the user can be obtained by a Global Positioning System receiver comprised within the wireless wearable electronic device 100 or within the other computing device 110 (e.g. in cases where the other computing device 110 is carried by the user, such as a smartphone, etc.).

Such presented data can enable the user wearing the wireless wearable electronic device 100 to understand the circumstances under which the information of block 310 was acquired by the sensors 230, such as the date and/or time of the day and/or the user's location and/or the user's participation in certain events, and/or the user's proximity to certain people/places and/or proximity of the stress score calculation time to a mental process such as meditation or mindfulness exercises performed by the user, etc. Having such understanding of the circumstances can enable a user to learn to avoid specific circumstances that have a negative effect on their stress level. For example, a user can learn that being around certain people increases his stress level, whereas certain types of meditation exercises reduce his level of stress.

It is to be noted that the stress monitoring process 300 can be performed repeatedly, e.g. continuously, according to a pre-determined schedule, according to a set of one or more rules, randomly throughout a given time period, etc. In some cases, it may be desired to repeat the process at random time periods so that the user wearing the wireless wearable electronic device 100 is unaware of the times at which a stress score is calculated. Such random operation can eliminate biases resulting from the user awareness of the measurement. In specific cases, the stress monitoring process 300 can be performed every hour, optionally at a random time during every such hour.

In some cases, prior to the sensors 230 acquiring the information obtained at block 310, the system 10 can be configured to obtain an indication of a motion status of the wireless wearable electronic device 100 and only if the indication indicates that the wireless wearable electronic device 100 is not moving—the sensors acquire the information. For this purpose, the wireless wearable electronic device 100 can comprise a motion sensor (e.g. one or more accelerometers, etc.) that can provide the indication of the motion status of the user wearing the wireless wearable electronic device 100.

It is to be noted that, with reference to FIG. 3, some of the blocks can be integrated into a consolidated block or can be broken down to a few blocks and/or other blocks may be added. Furthermore, in some cases, the blocks can be performed in a different order than described herein (for example, block 360 can be performed before block 340 and/or 350, block 370 can be performed before block 370, etc.). It is to be further noted that some of the blocks are optional. It should be also noted that whilst the flow diagram is described also with reference to the system elements that realizes them, this is by no means binding, and the blocks can be performed by elements other than those described herein.

FIG. 4 is a flowchart illustrating one example of a sequence of operations carried out for calculating a Heart Rate Variability (HRV) of a user of the stress level management system, in accordance with the presently disclosed subject matter.

According to some examples of the presently disclosed subject matter, system can be configure to perform an HRV determination process 300, e.g. utilizing the stress determination module 250.

For that purpose, system 10 can be configured to divide the given time period during which the information enabling determination of the HRV of the user wearing the wireless wearable electronic device 100 was acquired to a plurality of time windows (block 410). In some cases, each pair of subsequent time windows can partially overlap. In an example where the given time period is five minutes, the time windows can be, for example, nine time periods of sixty seconds, where each pair of subsequent time windows has a thirty seconds overlap.

The system then can calculate, for each time window, a corresponding HRV (that can be a HRV-HF and/or a HRV-LF, or a combined measure of both HRV-HF and HRV-LF), based on the information acquired by the sensors 230 during each corresponding time period (block 420). After calculating the HRVs for each time window, the system 10 can select the maximal HRV out of the calculated HRVs calculated at block 420, which is the HRV indicative of the lowest stress level of the user wearing the wireless wearable electronic device 100 during the given time period (block 430). Such maximal HRV is the HRV determined at block 320.

It is to be noted that when the wireless wearable electronic device 100 is worn on the user's body, movements of the body and/or certain positions the body assumes, can influence the measurements, and therefore, performing the HRV determination process 300 enables determining a stress score with an improved quality in comparison to other methods.

It is to be noted that, with reference to FIG. 4, some of the blocks can be integrated into a consolidated block or can be broken down to a few blocks and/or other blocks may be added. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A system comprising:
at least one sensor, comprised within a wireless wearable electronic device, the sensor configured to sense, over a given time period, information enabling determination of a Heart Rate Variability (HRV) of a wearer of the wireless wearable electronic device; and
one or more processing units configured to:
obtain, from the sensor, the information enabling determination of the HRV of the wearer;
determine, using the information, the HRV of the wearer;
calculate, utilizing the HRV, a stress score indicative of a stress level of the wearer.

2. The system of claim 1, wherein the HRF includes at least one of:
a. a High Frequency HRV (HRV-HF);
b. a Low Frequency HRV (HRV-LF);
c. a combined measure of the HRV-HF and the HRV-LF.

3. The system of claim 1, wherein at least one of the processing units is further configured to store the stress score and a time associated with the stress score in a data repository comprising a plurality of previously calculated stress scores, each indicative of a corresponding stress level of the wearer at a corresponding time.

4. The system of claim 3 wherein the system comprises at least one display and at least one of the processing units is further configured to retrieve at least one previously calculated stress score of the previously calculated stress scores from the data repository, and present the at least one previously calculated stress score on the display, optionally along with at least one corresponding environmental data associated therewith.

5. The system of claim 4 wherein the corresponding environmental data includes one or more of:
a. data indicative an event the wearer attended at the corresponding time of the corresponding previously calculated stress score;
b. data indicative of participants of the event; and
c. data indicative of a location of the wearer at the corresponding time of the corresponding previously calculated stress score.

6. The system of claim 1 wherein at least one of the processing units is further configured to alert the wearer when the stress score exceeds a threshold.

7. The system of claim 6 wherein the threshold is pre-defined or dynamically determined by at least one of the processing units utilizing previously calculated stress scores each indicative of a corresponding stress level of the wearer.

8. The system of claim 1 wherein the wireless wearable device further comprises a motion detection system configured to obtain an indication of a motion status of the wireless wearable device and wherein the information enabling determination of the HRV of the wearer is obtained when the indication indicates that the wireless wearable device is not moving.

9. The system of claim 8 wherein the motion detection system comprises at least one accelerometer.

10. The system of claim 1 wherein the determine comprises:
dividing the given time period to a plurality of time windows;
calculating, for each time window, a corresponding time window HRV; and
selecting the maximal time window HRV, indicative of the lowest stress level of the wearer during the given time window.

11. The system of claim 10 wherein each pair of subsequent time windows of the plurality of time windows at least partially overlap.

12. The system of claim 1 wherein the wireless wearable electronic device is worn on a wrist of the wearer.

13. The system of claim 1 wherein at least a first processing unit of the processing units is comprised within the wireless wearable electronic device, and at least a second processing units of the processing units is external to the wireless wearable electronic device.

14. The system of claim 1 wherein the stress score is calculated also utilizing at least one of the following parameters:
a. an average interval between heartbeats of the user during the given time period;
b. a standard deviation of intervals between heartbeats of the user during the given time period.

15. A method comprising:
obtaining from at least one sensor, comprised within a wireless wearable electronic device, information sensed during a given time period, the information enabling determination of a Heart Rate Variability (HRV) of a wearer of the wireless wearable electronic device;
determining, using the information, the HRV of the wearer;
calculating, utilizing the HRV, a stress score indicative of a stress level of the wearer.

16. The method of claim 15, wherein the HRF includes at least one of:
a. a High Frequency HRV (HRV-HF);
b. a Low Frequency HRV (HRV-LF);
c. a combined measure of the HRV-HF and the HRV-LF.

17. The method of claim 15, further comprising storing the stress score and a time associated with the stress score in a data repository comprising a plurality of previously calculated stress scores, each indicative of a corresponding stress level of the wearer at a corresponding time.

18. The method of claim 17 further comprising retrieving at least one previously calculated stress score of the previously calculated stress scores from the data repository, and presenting the at least one previously calculated stress score on a display, optionally along with at least one corresponding environmental data associated therewith.

19. The method of claim 18 wherein the corresponding environmental data includes one or more of:
a. data indicative an event the wearer attended at the corresponding time of the corresponding previously calculated stress score;
b. data indicative of participants of the event; and
c. data indicative of a location of the wearer at the corresponding time of the corresponding previously calculated stress score.

20. The method of claim 15 further comprising alerting the wearer when the stress score exceeds a threshold.

21. The method of claim 20 wherein the threshold is pre-defined or dynamically determined utilizing previously calculated stress scores each indicative of a corresponding stress level of the wearer.

22. The method of claim 15 further comprising obtaining, from a motion detection system comprised within the wireless wearable device, an indication of a motion status of the wireless wearable device and wherein the information enabling determination of the HRV of the wearer is obtained when the indication indicates that the wireless wearable device is not moving.

23. The method of claim 22 wherein the motion detection system comprises at least one accelerometer.

24. The method of claim 15 wherein the determining comprises:
dividing the given time period to a plurality of time windows;
calculating, for each time window, a corresponding time window HRV; and
selecting the maximal time window HRV, indicative of the lowest stress level of the wearer during the given time window.

25. The method of claim 24 wherein each pair of subsequent time windows of the plurality of time windows at least partially overlap.

26. The method of claim 15 wherein the wireless wearable electronic device is worn on a wrist of the wearer.

27. The method of claim 15 wherein the stress score is calculated also utilizing at least one of the following parameters:
a. an average interval between heartbeats of the user during the given time period;
b. a standard deviation of intervals between heartbeats of the user during the given time period.

28. A non-transitory computer readable storage medium having computer readable program code embodied therewith, the computer readable program code, executable by at least one processor of a computer to perform a method comprising:
obtaining from at least one sensor, comprised within a wireless wearable electronic device, information sensed during a given time period, the information enabling determination of a Heart Rate Variability (HRV) of a wearer of the wireless wearable electronic device;
determining, using the information, the HRV of the wearer;
calculating, utilizing the HRV, a stress score indicative of a stress level of the wearer.
